SEQUENCE LISTING (32 sequences)

SEQ ID NO: 1 - skipped

SEQ ID NO: 2 - protein, 16 aa, Homo sapiens
  UNSURE 1
  UNSURE 16
KAPQLTSSELMAITRK

SEQ ID NO: 3 - protein, 19 aa, Homo sapiens
  UNSURE 1
  UNSURE 19
KCCQGQEQEVCFAEEGQKL

SEQ ID NO: 4 - protein, 16 aa, Homo sapiens
  UNSURE 1
  UNSURE 16
KAPQLTSSELMAITRK

SEQ ID NO: 5 - protein, 22 aa, Homo sapiens
  UNSURE 1
  UNSURE 22
KCFQTENPLECQDKGEEELQKY

SEQ ID NO: 6 - protein, 36 aa, Homo sapiens
  UNSURE 1
  UNSURE 36
KDALTAIEKPTGDEQSSGCLENQLPAFLEELCHEKE

SEQ ID NO: 7 - protein, 12 aa, Homo sapiens
  UNSURE 1
  UNSURE 12
RDFNQFSSGEKN

SEQ ID NO: 8 - protein, 16 aa, Homo sapiens
  UNSURE 1
  UNSURE 16
KDLCQAQGVALQTMKQ

SEQ ID NO: 9 - protein, 16 aa, Homo sapiens
  UNSURE 1
  UNSURE 16
RESSLLNQHACAVMKN

SEQ ID NO: 10 - protein, 14 aa, Homo sapiens
  UNSURE 1
  UNSURE 14
RGDVLDCLQDGEKI

SEQ ID NO: 11 - protein, 24 aa, Homo sapiens
  UNSURE 1
  UNSURE 24
RGQCIIHAENDEKPEGLSPNLNRF

SEQ ID NO: 12 - protein, 23 aa, Homo sapiens
  UNSURE 1
  UNSURE 23
RHEMTPVNPGVGQCCTSSYANRR

SEQ ID NO: 13 - protein, 10 aa, Homo sapiens
  UNSURE 1
  UNSURE 10
RHNCFLAHKK

SEQ ID NO: 14 - protein, 17 aa, Homo sapiens
  UNSURE 1
  UNSURE 17
RHPFLYAPTILLWAARY

SEQ ID NO: 15 - protein, 13 aa, Homo sapiens
  UNSURE 1
  UNSURE 13
RHPQLAVSVILRV

SEQ ID NO: 16 - protein, 17 aa, Homo sapiens
  UNSURE 1
  UNSURE 17
KIMSYICSQQDTLSNKI

SEQ ID NO: 17 - protein, 17 aa, Homo sapiens
  UNSURE 1
  UNSURE 17
KKAPQLTSSELMAITRK

SEQ ID NO: 18 - protein, 28 aa, Homo sapiens
  UNSURE 1
  UNSURE 28
KKPTPASIPLFQVPEPVTSCEAYEEDRE

SEQ ID NO: 19 - protein, 34 aa, Homo sapiens
  UNSURE 1
  UNSURE 34
KKPTPASIPLFQVPEPVTSCEAYEEDRETFMNKF

SEQ ID NO: 20 - protein, 21 aa, Homo sapiens
  UNSURE 1
  UNSURE 21
KLLACGEGAADIIIGHLCIRH

SEQ ID NO: 21 - protein, 16 aa, Homo sapiens
  UNSURE 1
  UNSURE 16
KLVLDVAHVHEHCCRG

SEQ ID NO: 22 - protein, 17 aa, Homo sapiens
  UNSURE 1
  UNSURE 17
KMAATAATCCQLSEDKL

SEQ ID NO: 23 - protein, 15 aa, Homo sapiens
  UNSURE 1
  UNSURE 15
KNIFLASFVHEYSRR

SEQ ID NO: 24 - protein, 11 aa, Homo sapiens
  UNSURE 1
  UNSURE 11
KQEFLINLVKQ

SEQ ID NO: 25 - protein, 25 aa, Homo sapiens
  UNSURE 25
KQKPQITEEQLEAVIADFSGLLEKC

SEQ ID NO: 26 - protein, 18 aa, Homo sapiens
  UNSURE 1
  UNSURE 18
RRHPFLYAPTILLWAARY

SEQ ID NO: 27 - protein, 14 aa, Homo sapiens
  UNSURE 1
  UNSURE 14
RRHPQLAVSVILRV

SEQ ID NO: 28 - protein, 24 aa, Homo sapiens
  UNSURE 1
  UNSURE 24
RRPCFSSLVVDETYVPPAFSDDKF

SEQ ID NO: 29 - protein, 29 aa, Homo sapiens
  UNSURE 1
  UNSURE 29
RRPCFSSLVVDETYVPPAFSDDKFIFHKD

SEQ ID NO: 30 - protein, 11 aa, Homo sapiens
  UNSURE 1
  UNSURE 11
RTFQAITVTKL

SEQ ID NO: 31 - protein, 16 aa, Homo sapiens
  UNSURE 1
  UNSURE 16
KYGHSDCCSQSEEGRH

SEQ ID NO: 32 - protein, 12 aa, Homo sapiens
  UNSURE 1
  UNSURE 12
KYIQESQALAKR

(19) United States
(12) Patent Application Publication
(10) Pub. No.: US 2023/0296619 A1
(43) Pub. Date: Sep. 21, 2023

(54) Method for Detecting Alpha Fetoprotein Glycoforms by High Resolution Intact Mass Analysis

(71) Applicant: University of Utah Research Foundation, Salt Lake City, UT (US)

(72) Inventors: Yifei K. Yang, Salt Lake City, UT (US); Mark M. Kushnir, Salt Lake City, UT (US); Carmen A. Dunbar, Salt Lake City, UT (US)

(21) Appl. No.: 17/951,072

(22) Filed: Sep. 22, 2022

Related U.S. Application Data
(60) Provisional application No. 63/247,260, filed on Sep. 22, 2021.

(51) Int. Cl. G01N 33/68; C12N 15/115; G01N 33/574; G01N 33/577

(52) U.S. Cl. CPC G01N 33/6848; C12N 15/115; G01N 33/57476; G01N 33/577

(57) ABSTRACT

Methods for identifying and determining an abundance of an alpha fetoprotein (AFP) glycoform in a biological sample includes obtaining a biological sample, separating AFP from the biological sample as an enriched AFP sample, separating intact AFP from the enriched AFP sample, subjecting the intact AFP to high resolution mass spectrometry to generate a mass spectrum, identifying at least one AFP glycoform from the mass spectrum, and determining an abundance of the at least one AFP glycoform in the biological from the mass spectrum.

### SEQUENCE LISTING

This application contains a sequence listing which is incorporated herein by reference in ST.26 XML format named 00846-U7273.NP.xml, created Sep. 22, 2022, and is 36 KB in size. The sequences contained in the sequence listing are found throughout the originally filed application.

### BACKGROUND

Glycosylation is a post-translational modification (PTM) mechanism of proteins that modulates protein function and is implicated in protein folding, trafficking, and cell-to-cell communications. Dynamic changes in glycosylation states have been associated with different medical conditions, including cancer, viral infection responses, and Alzheimer's disease. In such cases, the extent of the glycosylation could be associated with the disease progression and prognosis.

Alpha fetoprotein (AFP) is an established circulating cancer biomarker implicated in multiple neoplastic malignancies, including hepatocellular carcinoma (HCC) and nonseminomatous testicular tumors. AFP is a glycoprotein predominantly expressed in the embryonic yolk sac and fetal liver, and its elevation as a circulating biomarker can be observed in liver cirrhosis, pregnancy, and in neonates. Mature AFP has a single consensus N-linked glycosylation site at Asparagine 251 (N251), and a high degree of structural variation in AFP glycan modifications have been identified in serum samples. There is limited evidence suggesting a possible O-glycosylation site within AFP based on cell culture study; however, no AFP O-glycosylation has been confirmed in authentic serum samples. In addition to glycosylation, six putative phosphorylation sites in AFP have been reported. The existence of different isoforms of post-translationally modified AFP have been found in patients with benign and malignant diseases.

### BRIEF DESCRIPTION OF THE DRAWINGS

FIG. 1A illustrates a glycan structure associated with a glycoform of AFP-L3 in accordance with an example embodiment;

FIG. 1B illustrates a glycan structure associated with a glycoform of AFP-L3 in accordance with an example embodiment;

FIG. 1C illustrates a glycan structure associated with a glycoform of AFP-L3 in accordance with an example embodiment;

FIG. 1D illustrates a glycan structure associated with a glycoform of AFP-L3 in accordance with an example embodiment;

FIG. 1E illustrates a glycan structure associated with a glycoform of AFP-L3 in accordance with an example embodiment;

FIG. 1F illustrates a glycan structure associated with a glycoform of AFP-L3 in accordance with an example embodiment;

FIG. 1G illustrates a glycan structure associated with a glycoform of AFP-L1 in accordance with an example embodiment;

FIG. 1H illustrates a glycan structure associated with a glycoform of AFP-L1 in accordance with an example embodiment;

FIG. 1I illustrates a glycan structure associated with a glycoform of AFP-L1 in accordance with an example embodiment;

FIG. 1J illustrates a glycan structure associated with a glycoform of AFP-L1 in accordance with an example embodiment;

FIG. 1K illustrates a glycan structure associated with a glycoform of AFP-L1 in accordance with an example embodiment;

FIG. 1L illustrates a glycan structure associated with a glycoform of AFP-L1 in accordance with an example embodiment;

FIG. 2A illustrates AFP separation from a biological sample in accordance with an example embodiment;

FIG. 2B shows a chromatogram of peaks of intact molecules of albumin and AFP in accordance with an example embodiment;

FIG. 2C shows deconvoluted masses of AFP from a multiply charged envelope in accordance with an example embodiment;

FIG. 3A provides a chromatogram showing separation of intact AFP in accordance with an example embodiment;

FIG. 3B shows a deconvoluted protein mass spectrum in accordance with an example embodiment;

FIG. 3C shows a collection of deconvoluted masses matched to full length AFP with different post-translational modification in accordance with an example embodiment;

FIG. 4 shows a deconvoluted mass spectrum of a peak eluted at retention time 9 min, corresponding to human serum albumin in patient serum samples after immune-enrichment and analyzation by LC-HRMS in accordance with an example embodiment;

FIG. 5A shows AFP glycoforms identified in a representative patient sample with relatively high AFP-L3% in accordance with an example embodiment;

FIG. 5B shows AFP glycoforms identified in a representative patient sample with relatively low AFP-L3% in accordance with an example embodiment;

FIG. 5C shows a distribution of relative abundances of various AFP proteoforms observed in analyzed authentic patient serum samples in accordance with an example embodiment;

FIG. 6 shows common glycan modifications available in the BioConfirm library in accordance with an example embodiment;

FIG. 7 shows a deconvoluted mass spectrum of AFP from a commercial standard;

FIG. 8 shows AFP-L1 and AFP-L3 glycoforms from authentic serum samples in accordance with an example embodiment;

FIG. 9 shows in accordance with an example embodiment;

FIG. 10 shows phosphorylation sites combined with 5 possible glycan modifications in accordance with an example embodiment; and

FIG. 11 shows a chromatogram of a patient serum sample with undetectable AFP in accordance with an example embodiment.

### DESCRIPTION OF EMBODIMENTS

Although the following detailed description contains many specifics for the purpose of illustration, a person of ordinary skill in the art will appreciate that many variations and alterations to the following details can be made and are considered included herein. Accordingly, the following embodiments are set forth without any loss of generality to, and without imposing limitations upon, any claims set forth. It is also to be understood that the terminology used herein is for describing particular embodiments only and is not intended to be limiting. Unless defined otherwise, all technical and scientific terms used herein have the same meaning as commonly understood by one of ordinary skill in the art to which this disclosure belongs. Also, the same reference numerals appearing in different drawings represent the same element. Numbers provided in flow charts and processes are provided for clarity in illustrating steps and operations and do not necessarily indicate a particular order or sequence.

Furthermore, the described features, structures, or characteristics can be combined in any suitable manner in one or more embodiments. In the following description, numerous specific details are provided, such as examples of layouts, distances, network examples, etc., to provide a thorough understanding of various embodiments. One skilled in the relevant art will recognize, however, that such detailed embodiments do not limit the overall concepts articulated herein but are merely representative thereof. One skilled in the relevant art will also recognize that the technology can be practiced without one or more of the specific details, or with other methods, components, layouts, etc. In other instances, well-known structures, materials, or operations may not be shown or described in detail to avoid obscuring aspects of the disclosure.

In this application, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the composition's nature or characteristics would be permissible if present under the “consisting essentially of” language, even though not expressly recited in a list of items following such terminology. When using an open-ended term in this written description, like “comprising” or “including,” it is understood that direct support should be afforded also to “consisting essentially of” language as well as “consisting of” language as if stated explicitly and vice versa.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a given term, metric, value, range endpoint, or the like. The degree of flexibility for a particular variable can be readily determined by one skilled in the art. However, unless otherwise expressed, the term “about” generally provides flexibility of less than 1%, and in some cases less than 0.01%. It is to be understood that, even when the term “about” is used in the present specification in connection with a specific numerical value, support for the exact numerical value recited apart from the “about” terminology is also provided.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 1.5, 2, 2.3, 3, 3.8, 4, 4.6, 5, and 5.1 individually.

This same principle applies to ranges reciting only one numerical value as a minimum or a maximum. Furthermore, such an interpretation should apply regardless of the breadth of the range, or the characteristics being described.

Reference throughout this specification to “an example” means that a particular feature, structure, or characteristic described in connection with the example is included in at least one embodiment. Thus, appearances of phrases including “an example” or “an embodiment” in various places throughout this specification are not necessarily all referring to the same example or embodiment.

The terms “first,” “second,” “third,” “fourth,” and the like in the description and in the claims, if any, are used for distinguishing between similar elements and not necessarily for describing a particular sequential or chronological order. It is to be understood that the terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” and the like refer to a property of a device, component, or activity that is measurably different from other devices, components, or activities in a surrounding or adjacent area, in a single device or in multiple comparable devices, in a group or class, in multiple groups or classes, or as compared to the known state of the art.

The term “authentic patient sample” is used to refer to AFP positive samples from individual patients, as opposed to, for example, an AFP negative patient sample spiked with AFP standards (i.e., a contrived/spiked sample).

An initial overview of embodiments is provided below, and specific embodiments are then described in further detail. This initial summary is intended to aid readers in understanding the disclosure more quickly and is not intended to identify key or essential technological features, nor is it intended to limit the scope of the claimed subject matter.

Observed correlations between different isoforms of post-translationally modified alpha fetoprotein (AFP) and certain medical conditions indicate that the specific glycosylation states of AFP can be utilized as biomarkers for disease monitoring and prognosis. Current techniques for measurement of clinically adopted cancer protein biomarkers, for example, are performed using affinity-based techniques. These methods can at times suffer from poor specificity and accuracy, and thus cannot distinguish between closely related proteoforms corresponding to different glycosylation modifications, which limit their diagnostic accuracy and value.

AFP glycoforms sharing a core fucosylation structure (AFP-L3), namely an α-linked fucose residue attached to the N-acetylglucosamine, are currently used clinically as a biomarker to predict the risk of developing hepatocellular carcinoma (HCC) and to monitor its recurrence in patients with impaired liver function. Commonly used methodology to measure the relative abundance of AFP-L3 glycoforms is lectin binding gel shift electrophoresis (GSE). Lens culinaris agglutinin (LCA) is a type of lectin that binds specifically to the core fucose moiety. Electrophoresis-based methods utilizing LCA binding can identify AFP-L3, however, they do not provide insights on structural variations of AFP glycoforms beyond the core fucosylation. In other words, existing methods using gel-shift electrophoresis can only identify differences in core fucosylation of various AFP glycoforms.

Several LC-MS/MS methods have been developed to profile and identify different glycoforms of AFP, which are based on quantification of AFP specific glycopeptides through digestion of proteins enriched from plasma or serum samples. These methods utilize glycopeptides without core fucosylation (AFP-L1) and with core fucosylation (AFP-L3) to calculate relative abundance of AFP-L3 glycoforms (AFP-L3%). Other studies also utilized enzyme and chemical treatment to release glycans from AFP and identify the glycan structure by electrospray ionization (ESI) and matrix-assisted laser desorption/ionization (MALDI) coupled with MS. Certain glycan structures require further derivatization or permethylation (a technique for the derivatization of carbohydrates to enhance ionization efficiency) prior to MS analysis due to their heat labile nature, which further increases the complexity of analysis and may affect the presently disclosed method's performance. Additionally, due to the enzymatic digestion prior to MS analysis, glycan structures from the associated AFP-L3 and AFP-L1 site can be truncated, thus precluding the characterization of the endogenously present AFP, AFP protein, AFP glycoforms, or other potential PTMs.

As described, elevated serum AFP can be observed in liver cirrhosis and HCC. The glycosylation patterns of AFP have been shown to differentiate such conditions, with AFP glycoforms with core fucosylation (AFP-L3) serving as a malignancy risk predictor for HCC. The inventors have developed a method to detect endogenously present AFP proteoforms and to quantify the relative abundance of AFP-L3 glycoforms (AFP-L3%) in serum samples and plasma samples. Based on the AFP profiles in authentic patient serum samples, the inventors have identified that the frequently observed AFP-L1 glycoforms G2S2, G2S1; and common AFP-L3 glycoforms are G2FS1 and G2FS2.

The method evaluation included reproducibility, specificity, dilution integrity, and comparison of the AFP-L3% with a lectin-binding gel shift electrophoresis (GSE) assay. The AFP-L1 and AFP-L3 proteoforms were reproducibly identified in multiple patient serum pools, resulting in reproducible AFP-L3% quantification. There was considerable agreement between the developed LC-HRMS and commercial GSE method when quantifying AFP-L3% (Pearson r=0.63).

The presently disclosed analytical method, in one example, accomplishes AFP glycoform analysis that utilizes AFP-specific enrichment followed by intact AFP liquid chromatography—high-resolution mass spectrometry (LC-HRMS) analysis to not only identify AFP with core fucosylation (AFP-L3) and without core fucosylation (AFP-L1), but to differentiate and characterize different glycoforms of AFP-L1 and AFP-L3. In some example uses, the masses of intact AFP proteoforms can be used to differentiate and identify post-translational modifications in their native states, beyond the glycan core fucosylation site. Furthermore, in other examples the presently disclosed method is used to make a quantitative determination as to the relative abundance of core fucosylated glycoforms (AFP-L3%) in a biological sample. In one nonlimiting example, the combined height of glycoforms in the deconvoluted spectrum are used to quantify AFP-L3% in each sample. This determination allows a simplified assay for predicting the risk associated with malignant hepatocellular carcinoma and/or other neoplasms associated with AFP elevation. In further examples, the method can be used to make a quantitative measurement of an individual AFP glycoforms or groups of AFP glycoforms. Compared to the electrophoretic and glycan, glycopeptide-based LC-MS/MS, or MALDI-MS approaches, the presently disclosed method can directly profile different AFP proteoforms, including glycoforms, with a simplified sample preparation, faster data acquisition, and easier data analysis.

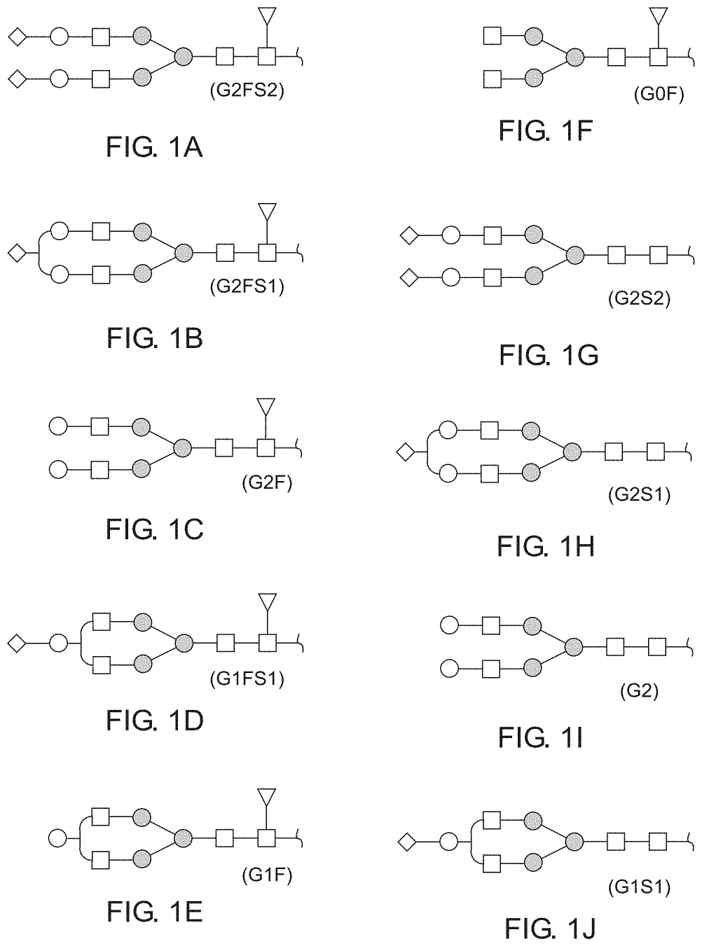

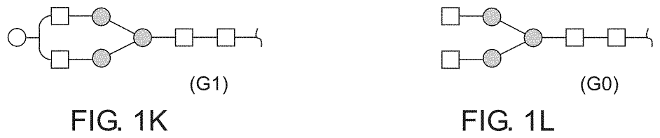

FIGS. 1A-1F show examples of the glycan structures associated with different glycoforms of AFP-L3 and FIGS. 1G-1L show examples of glycan structures associated with different glycoforms of AFP-L1. In these figures, squares represent the amide-derivative N-Acetylglucosamine (GlcNAc), triangles represent fucose, shaded circles represent mannose, unshaded circles represent galactose, and diamonds represent sialic acid (Neu5Ac). The presence of these and other glycoforms can be identified/differentiated by the present methods.

In one specific nonlimiting example, the method of identifying an AFP glycoform in a biological sample includes obtaining a biological sample, separating AFP from the biological sample as an enriched AFP sample, separating intact AFP from other residual non-specifically bound proteins in the enriched AFP sample, using chromatographic techniques, subjecting the intact AFP to high resolution mass spectrometry to generate a mass spectrum, and identifying the AFP glycoform or glycoforms from the mass spectrum.

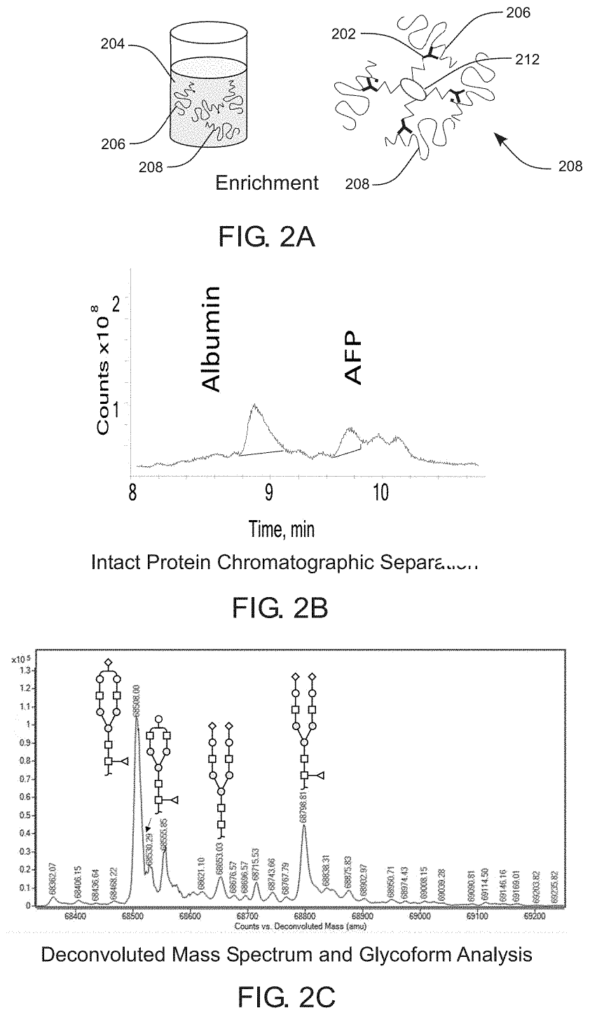

AFP can be separated by any affinity enrichment technique for protein separation known in the art. In one example, the AFP can be separated from the biological sample by any number of immune-enrichment techniques. Specifically, the AFP can be separated by antibody enrichment as is shown in FIG. 2A. In this case, anti-AFP antibody 202 is added to the biological sample 204 containing the AFP, which includes AFP-L3 glycoforms 206 and AFP-L1 glycoforms 208 to form antibody-bound AFP 210. FIG. 2A further shows AFP (AFP-L3 glycoforms 206 and AFP-L1 glycoforms 208) bound to anti-AFP antibody 202 conjugated to magnetic beads 212 to form the antibody-bound AFP 210. The magnetic beads 212 of the antibody-bound AFPs 210 are then magnetically separated from the biological sample 204, the beads are washed, and the AFP is eluted from the magnetic beads 212 as the enriched AFP sample (AFP-L3 glycoforms 206 and AFP-L1 glycoforms 208). It is noted that the eluted AFP glycoforms are intact AFP glycoforms having their unmodified glycans attached. In one more specific example of a monoclonal antibody enrichment, the magnetic beads were conjugated with anti-rabbit IgG antibody that can bind anti-AFP monoclonal rabbit antibody.

As noted above, the present enrichment phase of the disclosed methods is not limited to monoclonal antibody binding techniques. As another example, separating the AFP from the biological sample can be accomplished in a similar manner to that shown in FIG. 2A by using anti-AFP aptamers in lieu of anti-AFP monoclonal antibodies. Aptamers, which are well known in the art, are short single-stranded DNA or RNA molecules capable of selectively binding to a specific molecule, such as AFP. The aptamers have a sequence making them fold selectively around AFP. The aptamers can be conjugated to magnetic beads and used to enrich AFP though a similar technique as shown in FIG. 2A.

Following separation of the AFP from the biological sample, the intact AFP is separated from the enriched AFP sample. This separation can be accomplished by a variety of protein separation techniques, such as, for example, high performance liquid chromatography (HPLC), capillary electrophoresis, and the like. It is further noted that any liquid chromatography technique that achieves the necessary separation of AFP glycoforms can be used in the presently disclosed methods, including LC, HPLC, and other forms of liquid chromatography, such as reverse-phase HPLC (RP-HPLC) and the like.

In some examples, the intact AFP is separated from the enriched AFP sample and subjected to high resolution mass spectrometry to generate the mass spectrum via LC-HRMS. FIG. 2B shows an example of a chromatogram obtained in the analysis of an enriched neat serum sample (showing peaks of intact molecules of albumin and AFP). The data acquisition is performed using HRMS to generate high resolution mass signal spectra, differentiated from albumin. FIG. 2C shows deconvoluted masses of AFP from a multiply charged envelope, from which the AFP glycoforms are detected and identified. In some examples, the AFP-L3% is then quantified. An AFP glycoform can result in a plurality of peaks (FIG. 2C) from which the AFP glycoform can be identified by comparing a mass of at least one of the plurality of peaks with an expected mass of the AFP glycoform.

The present technology allows the characterization of specific glycoforms using LC-HRMS. Deconvoluted mass spectrum of intact AFP (i.e., the entire AFP glycoform) maintains the complex glycan structure that includes the AFP molecule and the glycan residues (fucosylated (AFP-L3) or nonfucosylated (AFP-L1)). As has been described above, the resulting AFP glycoform(s) can include any potential AFP glycoform moiety, including the AFP-L3 glycoforms shown in FIGS. 1A-1F and the AFP-L1 glycoforms shown in FIGS. 1G-1L.

In another example, a method of identifying and determining an abundance of an AFP glycoform in a biological sample includes obtaining a biological sample, separating AFP from the biological sample as an enriched AFP sample, separating intact AFP from the enriched AFP sample, separating intact AFP from other sample constituents using a chromatographic technique, subjecting the intact AFP to high resolution mass spectrometry to generate a mass spectrum, identifying at least one AFP glycoform from the mass spectrum, and determining the abundance of the at least one AFP glycoform in the biological sample from the mass spectrum.

Similar to the characterization method outlined above, the AFP can be separated by any affinity enrichment technique for protein separation known in the art. In one example, the AFP can be separated from the biological sample by any number of immune-enrichment techniques. Specifically, the AFP can be separated by antibody enrichment as is shown in FIG. 2A. In this case, anti-AFP antibodies 202 are added to the biological sample 204 containing the AFP, which includes AFP-L3 glycoforms 206 and AFP-L1 glycoforms 208 to form antibody-bound AFP 210. FIG. 2A further shows AFP (AFP-L3 glycoforms 206 and AFP-L1 glycoforms 208) bound to anti-AFP antibody 202 conjugated to magnetic beads 212 to form the antibody-bound AFP 210. The magnetic beads 212 of the antibody-bound AFPs 210 are then magnetically separated from the remainder of the biological sample 204 and the AFP is eluted from the magnetic beads 212 as the enriched AFP sample (AFP-L3 glycoforms 206 and AFP-L1 glycoforms 208). It is noted that the eluted AFP glycoforms are intact AFP glycoforms having their unmodified glycans attached. In one more specific example of a monoclonal antibody enrichment, the magnetic beads were conjugated with anti-rabbit IgG and can bind to include rabbit anti-AFP rabbit monoclonal antibodies. conjugated with anti-rabbit IgG.

As noted above, the present enrichment phase of the disclosed methods is not limited to monoclonal antibody binding techniques. As another example, separating the AFP from the biological sample can be accomplished in a similar manner to that shown in FIG. 2A by using anti-AFP aptamers in lieu of anti-AFP monoclonal antibodies. Aptamers, which are well known in the art, are short single-stranded DNA or RNA molecules capable of selectively binding to a specific molecule, such as AFP. The aptamers have a sequence making them fold selectively around AFP. The aptamers can be conjugated to magnetic beads and used to enrich AFP though a similar technique as shown in FIG. 2A.

Following separation of the AFP from the biological sample, the intact AFP is separated from the enriched AFP sample. This separation can be accomplished by a variety of protein separation techniques, such as, for example, liquid chromatography, reverse-phase high performance liquid chromatography, capillary electrophoresis, and the like.

In some examples, the intact AFP is separated from the enriched AFP sample, using chromatographic techniques, and subjected to high resolution mass spectrometry to generate the mass spectrum. FIG. 2B shows an example of data obtained for albumin and intact AFP by LC-HRMS. As is shown in FIG. 2B, the intact AFP and albumin are separated by liquid chromatography and the intact AFP product is analyzed by HRMS to generate mass spectrum signals of AFP. FIG. 2C shows the deconvolution of the AFP signals from multiple charge envelopes whereby the AFP glycoforms are detected. In some examples, the AFP-L3% is then quantified. An AFP glycoform can be identified by deconvoluting the mass spectrum into a plurality of peaks (FIG. 2C) and identifying the AFP glycoform by comparing a mass of at least one of the plurality of peaks with an expected mass of the AFP glycoform.

In one nonlimiting example, identifying the at least one AFP glycoform from the mass spectrum can include deconvoluting the mass spectrum into a plurality of peaks and identifying the AFP glycoform by matching a mass of at least one of the plurality of peaks with an expected mass of the AFP glycoform to confirm an AFP glycoform peak. Additionally, determining the abundance of the AFP glycoform further comprises measuring an intensity height of the AFP glycoform peak. In a more specific case, identifying the at least one AFP glycoform further includes identifying a plurality of AFP-L3 glycoform peaks from the mass spectrum to obtain a plurality of AFP-L3 glycoform peaks and determining the total abundance the AFP-L3 glycoforms in the biological sample by combining the intensity heights of all of AFP-L3 glycoform peaks.

FP-L1 glycoforms can be identified by matching masses of the peaks observed in the deconvoluted mass spectrum with the expected masses corresponding to the AFP-L1 and AFP-L3 glycoforms. Measuring the combined abundance of all peaks corresponding to AFP-L1 glycoforms determines the total abundance of AFP-L1 in the biological sample. Similarly, the combined abundance of all peaks corresponding to AFP-L3 glycoforms determines the total abundance of AFP-L1. The AFP-L3% can be readily calculated, in one nonlimiting example, from the total intensities of the total AFP-L3 and AFP-L1 glycoforms according to Formula I:

$$AFP - L 3 = \frac{total abundance of AFP - L 3 glycoform}{\begin{matrix}total abundance of AFP - L 3 glycoform + \\ total abundance of AFP - L 1 glycoform\end{matrix}} . \qquad Formula I .$$

### EXPERIMENTAL EXAMPLES

### Standards and Reagents

AFP purified from human umbilical cord blood was purchased from Lee Biosolutions (Maryland Heights, MO); monoclonal rabbit anti-AFP antibody and magnetic beads coated with anti-rabbit IgG antibodies were purchased from Thermo Fisher Scientific (Waltham, MA) and Eurofins Abraxis (Warminster, PA). Formic acid, glycine and 4-(2-hydroxyethyl)-1-piperazineethanesulfonic acid (HEPES) were purchased from MilliporeSigma (St Louis, MO); acetonitrile was purchased from Fisher Scientific (Waltham, MA), all other reagents were of the highest purity grade commercially available.

AFP Specific Immune Enrichment from Serum Samples:

500 μL of serum samples were aliquoted in 1.5 mL LoBind tubes (Eppendorf, Hamburg, Germany), 500 μL of HEPES buffer (pH 7.4), and 5 μL of anti-AFP antibody (Rabbit monoclonal antibody (3)) were added and the samples were incubated at 10° C. for 1 hour (with mixing, at 1100 rpm) in a Thermomixer (Eppendorf). After incubation, 5 μL of magnetic beads coated with anti-rabbit IgG antibody were added to the samples and the samples were incubated at 10° C. for 1 hour (with mixing at 1100 rpm). After incubation, the tubes were placed in a magnetic rack for 20 minutes to separate the magnetic beads from the supernatants and the supernatants were removed to waste. The magnetic beads were washed five times with 300 μL of phosphate buffered saline (PBS, pH 7.4, 20 mM). The washes included adding 300 μL of PBS to the tubes with the beads and vortexing the tubes for two minutes at 1100 rpm, followed by separation of the beads in magnetic rack (tubes were held in the rack for 5 minutes) and removal of the wash solution. After the last wash cycle, AFP was eluted from the magnetic beads with 75 μL glycine buffer (pH 2), the eluents were transferred into HPLC vials, and the samples were injected (20 μL) and subjected to LC-HRMS for intact protein analysis.

### Instrumental Analysis:

Instrumental analysis was performed using Q-TOF 6550 mass spectrometer, equipped with 1290 infinity I HPLC stack (Agilent Technologies, Santa Clara, CA). HPLC separation was performed using a 300Å, 3 μm, 2.1 mm×50 mm, polymeric reversed phase (PLRP) column (Agilent Technologies). Mobile phase A was 0.1% formic acid in water, and mobile phase B was 0.1% formic acid in acetonitrile, flow rate was 300 μL/min, mobile phase gradient was 80% to 40% A over 12 minutes, followed by column wash, conditioning, and re-equilibration. The column temperature was kept at 80° C. Data acquisition on the Q-TOF instrument was performed using high resolution, profile mode (4 GHz); the mass range was m/z 1,000-3,200 source conditions and acquisition parameters are listed Table 1.

TABLE 1
QTOF acquisition method settings.

| Parameter | Setting | |
|---|---|---|
| Gas Temp | 275° | C. |
| Drying Gas | 11 | l/min |
| Nebulizer | 35 | psi |
| Sheath Gas Temp | 400° | C. |
| Sheath Gas Flow | 11 | l/min |
| Vcap | 5500 | V |
| Nozzle Voltage | 500 | V |
| Fragmentor | 380 | V |
| Acquisition Rate | 3 | spectra/s |

| | |
|---|---|
| Mass Resolution at m/z 1000 | 35,000 |

### Intact Mass Data Analysis

Multiple charge envelopes corresponding to chromatographic peaks at the retention time (RT) of AFP and human albumin were selected for deconvolution using the maximum entropy algorithm in BioConfirm software 10.1 (Agilent Technologies). For AFP, the deconvolution was performed with target mass range between 68,000-80,000 Da using 7 Da mass step size. Deconvoluted masses at the expected AFP retention time were then matched to AFP full length sequence without its signal peptide (UniProt P02771|19-609; available at www.uniprot.org/uniprotkb/P02771/entry) and with 16 disulfide bonds. The variable modifications included known AFP glycan modifications and a maximum of six sites of phosphorylation. A small fraction of albumin was coenriched from patient serum samples along with AFP (most likely due to the cross-reactivity of the antibody); presence of the albumin peak and agreement of the deconvoluted mass with the expected mass of albumin, were used as an internal control to assess performance of the assay in every sample. Deconvolution of the peak at the retention time of albumin was performed using mass range 66,000-67,500 Da, with 7 Da step size.

Based on consensus results reported in previous literature, the allowed AFP glycoforms were: G0, G0F, G1, G1F, G1S1, G1FS1, G2, G2F, G2S2, G2S1, G2FS1, G2FS2 (FIGS. 1A-1L). These glycoforms also represent an unbiased and equal number of potential proteoforms representing the AFP-L1 and AFP-L3 isoforms. Match tolerance for the difference between the observed and the expected intact masses of the deconvoluted peaks was ±15 Da. For glycoforms G2FS1 and G2FS2, the mass matching tolerance was narrowed to ±5 Da due to the presence of interferences from unknown serum proteins observed in the deconvoluted mass spectra of AFP negative serum samples. Multiple deconvoluted masses within a ±1 Da window were summed up using their intensities as the abundance of the matched glycoform. Deconvoluted mass with the smallest delta relative to the theoretical target mass of each AFP proteoform was ruled in as the putative match. Based on the results observed in analysis of multiple patient samples and their abundances, the reproducible observed glycoforms (AFP-L1: G1, G1S1, G2, G2S1, G2S2, and AFP-L3: G1F, G1FS1, G2F, G2FS1, G2FS2; see FIGS. 1A-1L) were used to quantify the relative AFP-L3%. The relative AFP-L3% was calculated using Formula II:

$$AFP - L 3 \% = \frac{\sum Intensity Height of Identified L 3 isoforms}{\begin{matrix}\sum Intensity Height of Identified L 3 isoforms + \\ \sum Intensity Height of Identified L 1 isoforms\end{matrix}} \qquad Formula II .$$

### Immunoassay and GSE to Quantify of Total AFP and AFP-L3%

In this study we analyzed residual deidentified aliquots of patient serum samples (n=40) submitted for total AFP and AFP-L3% testing to ARUP Laboratories (Salt Lake City, UT). The AFP concentration was measured with Access AFP quantitative chemiluminescent immunoassay (Beckman Coulter, IN). The AFP-L3% was measured by a GSE method, the μTASWako AFP-L3 Immunological Test System (Wako Pure Chemical Industries, Osaka, Japan). In this method, LCA first recognizes and binds to the core fucosylation structure shared by AFP-L3 glycoforms; the LCA and AFP-L3 complex is further separated from AFP-L1 by gel electrophoresis; and an AFP-specific antibody with fluorescent label identifies and quantifies the relative abundance of AFP-L3 isoforms. The electrophoretic separation and fluorescence detection are both performed on a microfluidic chip.

All studies with human samples were approved by the Institutional Review Board of the University of Utah (Salt Lake City, UT). Patient serum specimens were measured by AFP immunoassay and GSE assay within one week of sample collection. The residual samples were stored at −20° C. prior to analysis by LC-HRMS within 6 months of sample collection.

### Method Evaluation

The method performance evaluation included the following experiments: analytical reproducibility, analytical specificity, dilution integrity, and comparison with lectin-binding GSE assay using authentic patient serum samples. The analytical reproducibility was evaluated by extraction and analysis of three AFP serum sample pools in four replicates; the samples contained different total AFP concentrations and different AFP-L3%. The specificity of the method was evaluated by analyzing five individual serum samples containing total AFP≤0.01 μg/mL. Dilution integrity and sensitivity of the method for detecting relative AFP-L3% were assessed by analysis of a dilution series of serum patient sample containing highly elevated AFP, diluted with AFP-negative serum sample, to create 4 samples containing progressively lower AFP concentration, while maintaining constant AFP-L3%.

### Statistical Analysis

Statistical analyses were performed using Excel (Microsoft) with additional modules from Analyse-IT, and JMP12 software (SAS Institute, USA).

### Discussion of Experimental Examples

### Immune-Enrichment Workflow and Intact Protein Analysis of AFP

Sample preparation was performed by enriching AFP from samples, followed by LC-HRMS analysis of the intact AFP (FIGS. 2A-2C). The assay includes an enrichment phase (FIG. 2A), which can include, without limitation, immune enrichment of endogenous AFP using rabbit anti-AFP monoclonal antibody and magnetic beads with conjugated anti-rabbit IgG, as shown, or any other type of enrichment such as, for example, aptamer enrichment (also referred to herein as “immune enrichment”). Enrichment further includes removal of non-specifically bound proteins by, in the monoclonal antibody enrichment, for example, washing the magnetic beads followed by elution of AFP from the beads. As is shown in FIG. 2B, the intact AFP is analyzed by LC-HRMS to generate a total ion chromatogram based on mass spectrum signals, followed by (FIG. 2C) deconvolution of AFP signals from multiple charge envelopes whereby the AFP proteoforms are detected. In some examples, the AFP-L3% is then quantified.

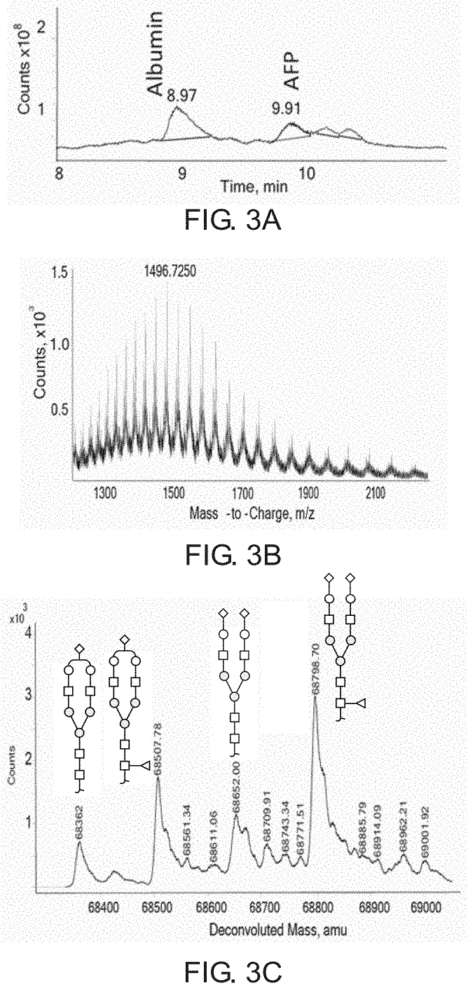

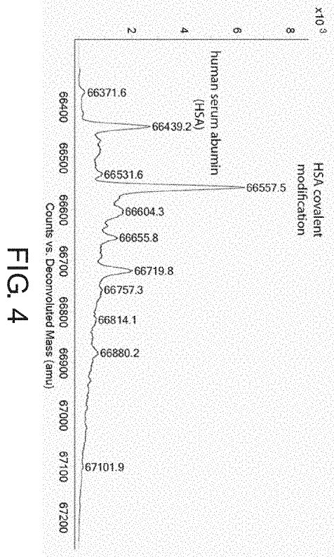

More specifically, following chromatographic separation, intact AFP was eluted at retention time (RT) of 9.9±0.2 min (FIG. 3A). From the deconvoluted intact protein mass spectrum (FIG. 3B), a collection of deconvoluted masses matched to full length AFP with different post-translational modifications was obtained (FIG. 3C). The chromatographic peak of human albumin was eluted ˜1 min earlier than the AFP peak (FIG. 3A and FIG. 4), and the albumin peak was present in all analyzed patient serum samples. FIG. 3A shows a total ion chromatogram of a patient serum sample containing 16.7 μg/mL of AFP. FIG. 4 shows a deconvoluted mass spectrum of peak eluted at retention time 9 min, corresponding to human serum albumin in patient serum samples after immune-enrichment and analyzed by LC-HRMS. The multiple charge envelopes at the respective retention times of AFP and albumin were selected (multiple charged states envelope at the retention time of the AFP peak (RT 9.9 min), is shown in FIG. 3B), the intact mass spectrum was deconvoluted and assigned to glycoforms of AFP (FIG. 3C and FIG. 4) and human albumin with covalent modifications. FIG. 4 shows a deconvoluted mass spectrum of human albumin (peak eluted at retention time 8.97 min, FIG. 3A) and various AFP glycoforms.

The efficiency of AFP immune-enrichment was assessed with authentic serum samples including both high and low AFP-L3%. It was observed that the enrichment recovery (absolute recovery of 30%) was independent of AFP isoform compositions. The specificity of the intact AFP enrichment was further confirmed by bottom-up analysis of AFP, in a workflow utilizing the same intact AFP enrichment method, followed by protein reduction, alkylation, tryptic digestion, and LC-MS/MS analysis. In this experiment, nine patient serum samples (mean/median AFP concentrations of 60.8/18.1 μg/mL and a commercial AFP standard (Lee Biosolutions)) were prepared and analyzed, using data dependent acquisition method on QTOF instrument. 32 unique peptides from AFP were detected in these samples, representing 69.9% AFP sequence coverage, as is shown in Table 2 (SEQ ID 01-SEQ ID 32). The data analysis and peptide identification were performed using Spectrum Mill B 06.00 (Agilent Technologies).

TABLE 2
Tryptic peptides of AFP

| | | Retention | MH⁺ | MH⁺ |
|---|---|---|---|---|
| | Peptide Sequence | time, min | matched, Da | error, Da |
| SEQ ID 01 | (K)AENAVECFQTK(A) | 7.53 | 1296.5889 | 0.0004 |
| SEQ ID 02 | (K)APQLTSSELMAITR(K) | 11.23 | 1517.7992 | 0.0022 |
| SEQ ID 03 | (K)CCQGQEQEVCFAEEGQ | 7.47 | 2086.8263 | 0.0023 |
| | K(L) | | | |
| SEQ ID 04 | (K)CFQTENPLECQDK(G) | 8.85 | 1668.6992 | 0.0038 |
| SEQ ID 05 | (K)CFQTENPLECQDKGEEELQ | 8.95 | 2482.0861 | 0.0071 |
| | K(Y) | | | |
| SEQ ID 06 | (K)DALTAIEKPTGDEQSSGCLE | 14.43 | 3829.7895 | 0.0143 |
| | NQLPAFLEELCHEK(E) | | | |
| SEQ ID 07 | (R)DFNQFSSGEK(N) | 7.75 | 1158.5062 | 0.0012 |
| SEQ ID 08 | (K)DLCQAQGVALQTMK(Q) | 9.52 | 1562.7665 | 0.0021 |
| SEQ ID 09 | (R)ESSLLNQHACAVMK(N) | 8.48 | 1587.7618 | 0.0032 |
| SEQ ID 10 | (R)GDVLDCLQDGEK(I) | 9.73 | 1348.6049 | 0.0026 |
| SEQ ID 11 | (R)GQCIIHAENDEKPEGLSPNL | 8.45 | 2491.1994 | 0.008 |
| | NR(F) | | | |
| SEQ ID 12 | (R)HEmTPVNPGVGQCCTSSYA | 6.7 | 2365.0118 | 16.003 |
| | NR(R) | | | |
| SEQ ID 13 | (R)HNCFLAHK(K) | 5.85 | 1026.4938 | 0.0005 |
| SEQ ID 14 | (R)HPFLYAPTILLWAAR(Y) | 16.12 | 1768.9897 | 0.0025 |
| SEQ ID 15 | (R)HPQLAVSVILR(V) | 11.2 | 1232.7474 | 0.0025 |
| SEQ ID 16 | (K)IMSYICSQQDTLSNK(I) | 9.13 | 1787.8302 | 0.0027 |
| SEQ ID 17 | (K)KAPQLTSSELMAITR(K) | 10.55 | 1645.8942 | 0.0023 |
| SEQ ID 18 | (K)KPTPASIPLFQVPEPVTSCEA | 12.95 | 2960.4346 | 0.0065 |
| | YEEDR(E) | | | |
| SEQ ID 19 | (K)KPTPASIPLFQVPEPVTSCEA | 13.07 | 3710.7717 | 0.0147 |
| | YEEDRETFMNK(F) | | | |
| SEQ ID 20 | (K)LLACGEGAADIIIGHLCIR(H) | 13.97 | 2052.0729 | 0.0033 |
| SEQ ID 21 | (K)LVLDVAHVHEHCCR(G) | 8.35 | 1744.837 | 0.0057 |
| SEQ ID 22 | (K)mAATAATCCQLSEDK(L) | 5.9 | 1656.7026 | 15.9975 |
| SEQ ID 23 | (K)NIFLASFVHEYSR(R) | 13.47 | 1582.8013 | 0.0038 |
| SEQ ID 24 | (K)QEFLINLVK(Q) | 12.92 | 1103.6459 | 0.0022 |
| SEQ ID 25 | (K)QKPQITEEQLEAVIADFSGL | 17.17 | 2586.3661 | 0.0049 |
| | LEK(C) | | | |
| SEQ ID 26 | (R)RHPFLYAPTILLWAAR(Y) | 15.05 | 1925.0908 | 0.0032 |
| SEQ ID 27 | (R)RHPQLAVSVILR(V) | 10.42 | 1388.8485 | 0.0014 |
| SEQ ID 28 | (R)RPCFSSLVVDETYVPPAFSD | 12.47 | 2529.1966 | 0.0073 |
| | DK(F) | | | |
| SEQ ID 29 | (R)RPCFSSLVVDETYVPPAFSD | 13.4 | 3201.5714 | 0.01 |
| | DKFIFHK(D) | | | |
| SEQ ID 30 | (R)TFQAITVTK(L) | 8.72 | 1008.5724 | 0.0006 |
| SEQ ID 31 | (K)YGHSDCCSQSEEGR(H) | 3.2 | 1671.6122 | 0.0014 |
| SEQ ID 32 | (K)YIQESQALAK(R) | 6.75 | 1150.6103 | 0.0006 |

### AFP Glycoform Profiles in the Authentic Patient Serum Samples

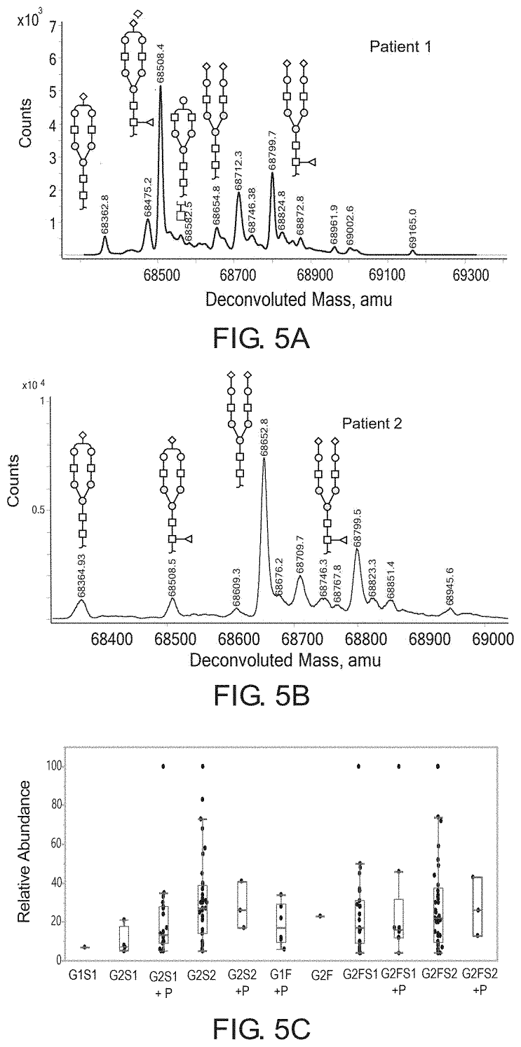

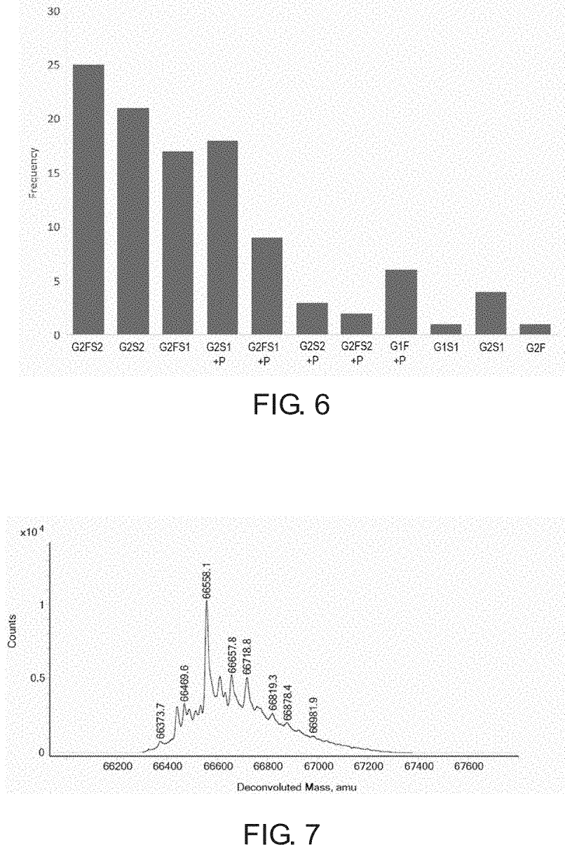

Masses of intact AFP glycoforms observed in deconvoluted mass spectra from analysis of authentic patient serum samples were matched to theoretical intact masses of the full-length AFP containing various glycosylation isoforms (FIG. 3C and FIGS. 1A-1L). In the initial proteoform profiling and assignment process, all common glycan modifications available in the BioConfirm library (Agilent Technologies) were allowed. Several AFP-L1 and AFP-L3 glycoforms were frequently observed in the authentic serum samples (FIG. 5C). The list of the allowed AFP glycoforms (FIG. 6) was further expanded to include equal numbers of glycoforms corresponding to the AFP-L1 and AFP-L3. One example of a glycoform list included 12 glycan modifications: G0, G0F, G1, G1F, G1S1, G1FS1, G2, G2F, G2S2, G2S1, G2FS1, G2FS2. Using the presently disclosed methodology, commercial AFP standard purified from human umbilical cord blood was analyzed. The deconvoluted mass spectrum of AFP from the commercial standard showed a distinctively different intact mass spectrum (FIG. 7), suggesting different proteoform features in umbilical cord blood as compared to pathologic serum samples from adults (FIGS. 5A-5B, for example).

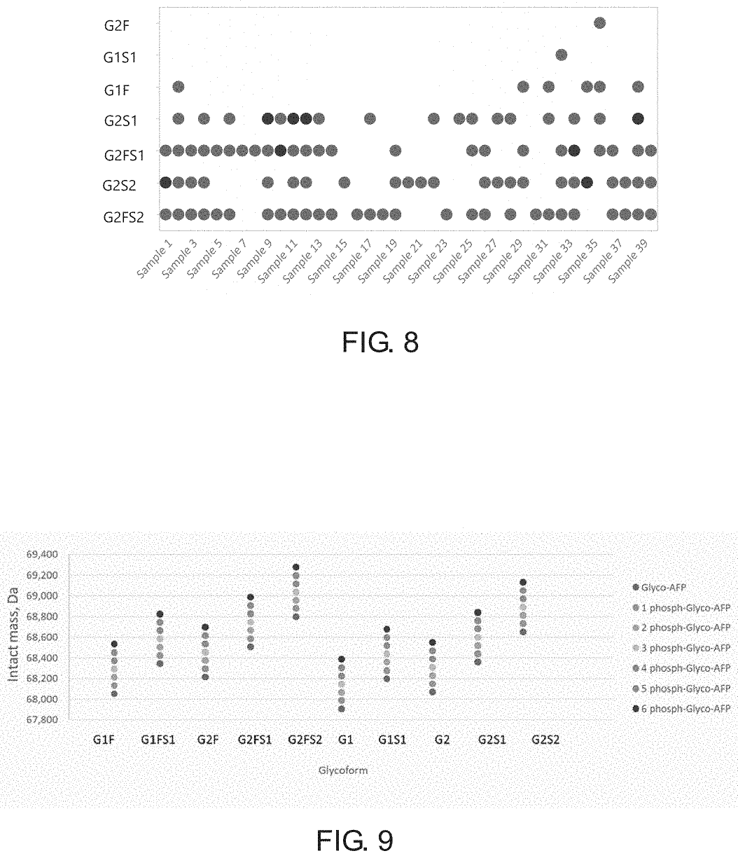

Out of all analyzed patient samples (n=40), AFP-L3 isoform G2FS2 (mass 68,799 Da) was most common (observed in 62.5% of the patient samples, Table 3, FIG. 6 and FIG. 8). The most frequently observed AFP-L1 isoform was G2S2 (mass 68,652 Da, observed in 52.5% of all samples). Both G2FS2 and G2S2 are complex glycan structures with two terminal sialic acids, which have been reported as glycan modifications in other studies based on bottom-up proteomics methods for AFP. Another frequently observed AFP-L3 isoform, G2FS1 (mass 68,508 Da), was identified in 42.5% of patient samples (Table 3 and FIG. 8). The presence of G2FS2 and G2FS1 are strongly associated with each other in patient samples (FIG. 5C and FIG. 8). FIG. 5A shows AFP glycoforms identified in a representative patient sample with relatively high AFP-L3%. FIG. 5B shows AFP glycoforms identified in a representative patient sample with relatively low AFP-L3%. FIG. 5C shows a distribution of relative abundances of various AFP proteoforms observed in analyzed authentic patient serum samples (n=40). G2S1 glycosylation (mass 68,841 Da) was another frequently observed AFP-L1 isoform (45% of patient samples; Table 3 and FIG. 6).

### AFP Phosphorylation in Patient Serum Samples

In addition to glycosylation, phosphorylation was an additional post-translational modification allowed in intact mass analysis and proteoform identification. Six putative phosphorylation sites in AFP sequence have been reported; however, no publications describe the occupancy of these phosphorylation sites. Variable phosphorylation sites combined with 5 possible glycan modifications can result in numerous closely spaced theoretical target masses (FIG. 9), hence creating large numbers of matches for some of the deconvoluted masses. FIG. 9 shows all possible AFP proteoforms and their expected intact masses with variable phosphorylation (up to six) and 10 glycan modifications. To minimize these potential matches, the identification criteria was narrowed to allow two occupancy states: 0 or 6 phosphorylation. In addition to non-phosphorylated forms (glycans G2S1, G2S2, G1F, G2FS1, and G2FS2), corresponding proteoforms with 6 phosphorylation sites (mass 68,841 Da, 69,132 Da, 68,534 Da, 68,987 Da, and 69,278 Da respectively) were used to quantify AFP-L3%. The identified proteoforms exhibited variable relative abundance among the analyzed patient samples (FIG. 5C).

TABLE 3
Frequently observed AFP proteoforms and their detection
frequencies in 40 analyzed patient samples.

| | Occurrence | | | |
|---|---|---|---|---|
| | Frequencies | | | |
| | (Number of | Glycan | | Glycoform |
| Intact Mass | patients | Modifi- | | Classi- |
| (Da) | observed) | cations | Phosphorylation | fication |
| 68,361 ± 15 | 4 | G2S1 | None | L1 isoform |
| 68,508 ± 5 | 17 | G2FS1 | None | L3 isoform |
| 68,534 ± 15 | 6 | G1F | 6 phosphorylation | L3 isoform |
| 68,799 ± 5 | 25 | G2FS2 | None | L3 isoform |
| 68,652 ± 15 | 21 | G2S2 | None | L1 isoform |
| 68,841 ± 15 | 18 | G2S1 | 6 phosphorylation | L1 isoform |
| 68987 ± 15 | 9 | G2FS1 | 6 phosphorylation | L3 isoform |

### Method Performance for Determining AFP-L3%:

Specificity. Serum samples containing less than 0.01 μg/mL of AFP (determined by immunoassay) were analyzed using the presently disclosed intact AFP LC-HRMS method to assess specificity of the analysis. The absence of AFP in the serum samples was corroborated by the absence of AFP chromatographic peak at the expected retention time. FIG. 11 shows the chromatogram of a patient serum sample with undetectable AFP (AFP concentration determined by Access AFP immunoassay, Beckman Coulter; LOQ of the assay 0.001 μg/mL). Expected RT for AFP is 9.9 min. To assess method specificity, background MS intensity within ±0.5 min of the expected RT window of AFP was deconvoluted and the intact mass spectra were inspected for presence of the expected intact masses corresponding to the monitored AFP proteoforms. Deconvoluted intact masses in the vicinity of 68,550 Da and 68,679 Da were observed in few of the AFP negative serum samples. Based on the above observations, the two possible proteoforms, phosphorylated AFP-G2 (mass 68,550 Da) and AFP-G1S1 with (mass 68,679 Da), were excluded from the target matches list, due to potential false-identification. Since both proteoforms belong to the AFP-L1 isoform, the corresponding AFP-L3 glycoforms (G2F, G1FS1) with phosphorylation (mass 68,696 Da, 68,825 Da) were also excluded from the potential target list.

Reproducibility. To evaluate assay reproducibility in quantifying AFP-L3%, three serum pools at different AFP concentrations (Table 4) prepared and analyzed by mixing patient serum samples containing high concentrations of AFP with AFP-negative serum samples. Table 4 shows the reproducibility in quantifying AFP-L3% based on replicate analysis of serum pools. Four replicate extractions and subsequent analysis were performed for each pool and AFP-L3% were calculated using the height of deconvoluted masses of detected glycoforms (Table 4). The variation in AFP-L3% among the replicate analyses was calculated and expressed as coefficient of variation (CV%). In two serum pools with similar total AFP concentration, but different AFP-L3% (pool 1 and 2), the imprecision was 1.6% and 17.7% (Table 4). In sample pools 1 and 2, different sets of AFP glycoforms were detected, potentially impacting the reproducibility in quantifying AFP-L3% in these samples. The highest AFP concentration pool 3 (66.4 μg/mL) exhibited consistent glycoform intensities across all four replicates (Table 4). The technical replicates based on reinjection of individual patient samples after immune-enrichment showed reproducible performance when quantifying AFP-L3% (Table 5). Table 5 shows the reproducibility of AFP-L3% based on replicate injections (N=2) of patient serum samples after AFP immune-enrichment (instrumental analysis was performed on the day of the sample preparation (1^{st} injection) and after a week of storage (2^{nd} injection).

TABLE 4
Reproducibility in quantifying AFP-L3% based
on replicate analysis of serum pools.

| Serum Pools | AFP concentration (μg/mL) | Average AFP-L3% | CV % |
|---|---|---|---|
| Pool 1 | 16.7 | 70.8% | 1.6% |
| Pool 2 | 17.0 | 55.8% | 17.7% |
| Pool 3 | 66.4 | 55.3% | 6.9% |

TABLE 5
Reproducibility of AFP-L3% based on replicate injections (N = 2).

| | | Sum of AFP-L1 | | Sum of AFP-L3 | | | |
|---|---|---|---|---|---|---|---|
| | Total | proteoforms | | proteoforms | | AFP-L3, % | |
| | AFP, | 1^{st} | 2^{nd} | 1^{st} | 2^{nd} | 1^{st} | 2^{nd} |
| Sample ID | μg/mL | injection | injection | injection | injection | injection | injection |
| Sample P1 | 13.9 | 3.5E+04 | 1.0E+04 | 2.8E+05 | 2.5E+05 | 89 | 96 |
| Sample P2 | 39.9 | 1.6E+05 | 8.1E+04 | 1.1E+05 | 4.5E+04 | 41 | 36 |
| Sample P3 | 276 | 1.7E+05 | 5.6E+04 | 5.8E+04 | 2.6E+04 | 26 | 32 |
| Sample P4 | 22.3 | 0.0E+00 | 0.0E+00 | 4.1E+04 | 1.0E+04 | 100 | 100 |

Dilution integrity. Samples containing high and low concentration of AFP were mixed at different proportions to create four AFP pools with varying total AFP concentrations (115-450 μg/mL), while the same AFP-L3% (Table 6). Based on the LC-HRMS intact protein analysis, the most prominent glycoforms identified in these samples were G2FS2 and G2FS1 (both AFP-L3 isoforms). The mean measured AFP-L3% was consistent across the four pools (mean 87.1%; range 81.4-92.2%, Table 6).

TABLE 6
Dilution integrity of AFP-L3%.

| Serum Pools (varying AFP | | |
|---|---|---|
| proportions) | AFP concentration (μg/mL) | AFP-L3% |
| 100% | 456.5 | 81.4% |
| 75% Pool | 342.4 | 88.6% |
| 50% Pool | 228.3 | 92.2% |
| 25% Pool | 114.1 | 86.3% |

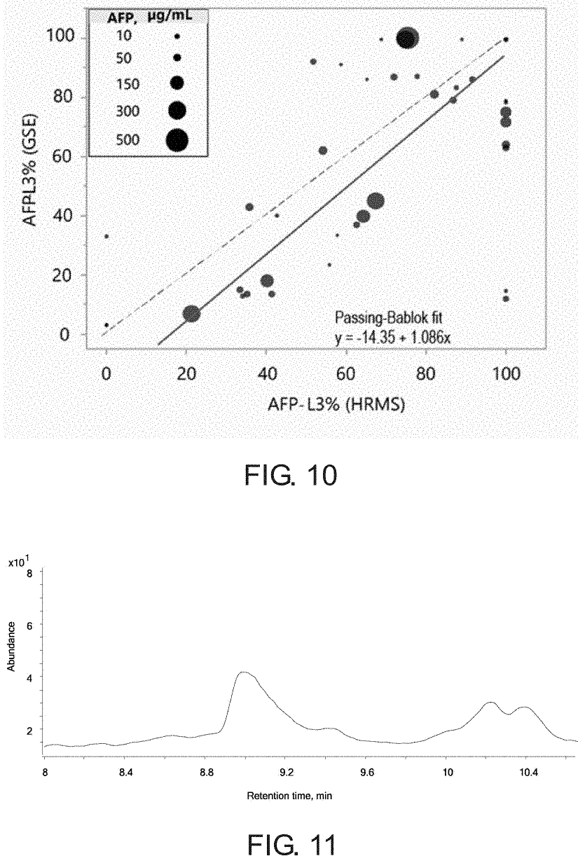

Comparison with the lectin based GSE method. As part of the method evaluation, 40 individual authentic serum samples were analyzed using the described method. The total AFP concentrations in these serum samples (determined by immunoassay) ranged between 6-450 μg/mL (mean /median of 87.2/37.2 μg/mL). The age of the participants ranged 48-91 years (mean/median of 65/64 years). Based on the AFP intact LC-HRMS methodology, the relative AFP-L3% isoforms in these specimens ranged from 0% (only AFP-L1 isoforms identified) to 100% (only AFP-L3 isoforms identified) (FIG. 10). FIG. 10 shows a comparison and correlation of AFP-L3% results based on LC-HRMS and GSE (Wako) in authentic serum samples (Pearson's r=0.645). The majority of the samples contained both AFP-L1 and AFP-L3 glycoforms (FIG. 8). The AFP-L3% quantified by LC-HRMS was compared to the AFP-L3% values determined by a GSE method (Wako). The two methods are comparable (Pearson's r=0.645, FIG. 10), while GSE method quantifies AFP-L3% with a noticeable positive proportional bias (8.6%, FIG. 10). The greater discrepancies were observed in patient specimens with predominantly AFP-L1 isoform and lower total AFP concentrations.

Here is reported a rapid and reproducible intact AFP LC-HRMS method to detect and identify glycoforms of AFP, a clinical biomarker to monitor impaired liver function. AFP proteoforms of reproducible and specific signals were selected to quantify AFP-L3%, a malignancy risk biomarker. To measure AFP-L3%, an intact protein analysis workflow (FIG. 2A-2C, for example) was established to determine the relative abundance of a subset of AFP glycoforms with and without core fucosylation. The methodology can be used in an assay for the HCC biomarker, AFP-L3%.

### Optimization of AFP Enrichment Process for Intact Protein Analysis

Due to the highly complex composition of human serum, immune enrichment techniques, such as antibody-based sample preparation, is utilized to selectively enrich AFP and to remove the majority of unrelated proteins prior to LC-HRMS analysis. In addition to AFP, the immune-enrichment recovered a small fraction of albumin, which can be seen in all analyzed serum samples after immune-enrichment. A likely explanation for the albumin presence is the partial homology between AFP and albumin, resulting in a certain degree of cross-reactivity with the anti-AFP antibody and a small fraction (˜0.05%) of the endogenously present albumin being coenriched with AFP.

Compared to glycopeptide based bottom-up methods to profile AFP glycoforms, the advantages of the present intact protein workflow include: (i) the immune-enrichment process is performed under physiologic conditions, which should preserve native AFP proteoforms; (ii) the sample preparation is faster and less complex, compared to the digestion based methods; and (iii) comparable ionization efficiency of different AFP proteoforms enables AFP-L3% determination without the need to quantify the absolute concentrations of targeted glycoforms. In agreement with glycopeptide-based methods, the most abundant and frequently observed AFP-L1 and AFP-L3 proteoforms contain complex glycan structures, with one or two terminal sialic acids. Compared to intact protein analysis, the released glycan analysis of AFP requires additional steps of glycan release, derivatization and permethylation after immune enrichment, an even more complex workflow.

### Optimization of LC-HRMS and Data Analysis Workflow

Chromatographic separation utilizing the 300 Å PLRP column was optimized to achieve AFP peak separation from other coenriched proteins such as albumin, and to ensure reproducible detection of AFP using deconvolution of the multiple charged envelopes within the AFP RT window. For MS detection, the mass range m/z 1000-3000 was sufficient to capture multiple charged envelopes of AFP and albumin and to minimize interferences from other proteins or small molecules.

The data analysis workflow was optimized to reduce computation time and to simplify the glycoform identification process. A moderate step size of 7 Da was selected to provide a reliable mass resolution in the deconvoluted mass spectra and high confidence matches to the expected AFP proteoforms with defined glycan and phosphorylation modifications. Based on the intact mass differences between AFP and human albumin, two discrete mass ranges, 68,000-80,000 Da and 66,000-67,500 Da respectively, were devised for deconvolution to obtain adequate quality mass spectra. Additionally, the mass matching tolerance was optimized using acquired data from enriched AFP positive patient samples. A narrow mass matching window of ±5 Da was initially utilized, but several deconvoluted masses of significant intensity were not identified as AFP glycoforms by the BioConfirm software, while they were clearly in the vicinity of the masses of the expected AFP with various glycan modifications. Evaluation of the larger tolerance window demonstrated that mass tolerance of ±15 Da enabled adequate identification of the targeted AFP glycoforms without introducing additional false-positive matches.

### Altered Glycosylation and Other Post-Translational Modifications in Cancers

Glycan synthesis and conjugation to proteins is a dynamic process that is impacted by changes in available sugar precursors, enzymes, and intracellular signal processes. In neoplasm conditions, the associated glycosylation changes have been shown to alter core fucosylation, increase α-2-6-sialylation, and increase N-glycan branching. When using LC-HRMS to profile intact AFP protein glycoforms, a diverse AFP glycoform distribution was observed in a majority of the analyzed patient samples (Table 3, FIG. 5A-C, and FIG. 8), these glycoforms were also observed in previous glycomics studies that profile AFP isoforms. In addition to the core fucosylation, the present study also showed that the most abundant and frequently observed AFP glycoforms detected are heavily sialylated, regardless of the core fucosylation status in the patient samples (FIG. 3C and FIG. 8). Because clinical diagnoses associated with the analyzed patient specimens were not available at the time of the method evaluation, the association between the degree of sialylation and hepatic malfunctions related to different pathological conditions was not assessed. In previous bottom-up study, however, it was reported that approximately half of the glycans identified in samples of HCC patients were sialylated, suggesting that sialylation in HCC is common, as sialylated carbohydrates play a role in cellular recognition, cell adhesion and cell signaling.

Although AFP phosphorylation has not been investigated extensively at a secretory protein level, our study revealed that a significant proportion of the AFP is phosphorylated. A previous proteomics study has identified six putative phosphorylation sites on the AFP protein. Phosphorylation is involved in regulation of cell function, including cell growth, differentiation, and apoptosis, with multiple signaling pathways participating in phosphorylation cascade; alterations in phosphorylation pathways have been shown to be associated with cancers.

### EXAMPLES

The following examples pertain to specific embodiments and point out specific features, elements, or steps that can be used or otherwise combined in achieving such embodiments.

A method of identifying an alpha fetoprotein (AFP) glycoform in a biological sample includes obtaining a biological sample, separating AFP from the biological sample as an enriched AFP sample, separating intact AFP from the enriched AFP sample, subjecting the intact AFP to high resolution mass spectrometry to generate a mass spectrum, and identifying an AFP glycoform from the mass spectrum.

The method of identifying an AFP glycoform in a biological sample, wherein identifying the AFP glycoform further includes deconvoluting the mass spectrum into a plurality of peaks and identifying the AFP glycoform by comparing a mass of at least one of the plurality of peaks with an expected mass of the AFP glycoform.

The method of identifying an AFP glycoform in a biological sample,
wherein separating the AFP from the biological sample further includes adding anti-AFP antibodies to the biological sample to form antibody-bound AFP, separating the antibody-bound AFP from the biological sample, and eluting the AFP from the anti-AFP antibodies as the enriched AFP sample.

The method of identifying an AFP glycoform in a biological sample wherein the anti-AFP antibodies are monoclonal antibodies.

The method of identifying an AFP glycoform in biological sample wherein separating the AFP from the biological sample further includes adding anti-AFP aptamers to the biological sample to form aptamer-bound AFP, separating the aptamer-bound AFP from the biological sample, and eluting the AFP from the anti-AFP aptamers as the enriched AFP sample.

The method of identifying an AFP glycoform in biological sample wherein separating the AFP from the biological sample, wherein the intact AFP is separated from the enriched AFP sample using high performance liquid chromatography.

The method of identifying an AFP glycoform in biological sample wherein separating the AFP from the biological sample, wherein the intact AFP is separated from the enriched AFP sample using capillary electrophoresis.

The method of identifying an AFP glycoform in biological sample wherein separating the AFP from the biological sample, wherein the high-resolution mass spectrometry is performed on the intact AFP with unmodified glycans.

The method of identifying an AFP glycoform in biological sample wherein separating the AFP from the biological sample, wherein the AFP glycoform is a glycoform variant of core fucosylated (AFP-L3).

The method of identifying an AFP glycoform in biological sample wherein separating the AFP from the biological sample, wherein the glycoform variant is selected from G1F, G1FS1, G2F, G2FS1, G2FS2, or a combination thereof.

The method of identifying an AFP glycoform in biological sample wherein separating the AFP from the biological sample, wherein the AFP glycoform is a glycoform variant of core nonfucosylated (AFP-L1).

The method of identifying an AFP glycoform in biological sample wherein separating the AFP from the biological sample, wherein the glycoform variant is selected from G1, G1S1, G2, G2S1, G2S2, or a combination thereof.

A method of identifying and determining an abundance of an alpha fetoprotein (AFP) glycoform in a biological sample includes obtaining a biological sample, separating AFP from the biological sample as an enriched AFP sample, separating intact AFP from the enriched AFP sample, subjecting the intact AFP to high resolution mass spectrometry to generate a mass spectrum, identifying at least one AFP glycoform from the mass spectrum, and determining an abundance of the at least one AFP glycoform in the biological from the mass spectrum.

A method of identifying and determining an abundance of an AFP glycoform, wherein identifying the at least one AFP glycoform from the mass spectrum further comprises deconvoluting the mass spectrum into a plurality of peaks and identifying the AFP glycoform by matching a mass of at least one of the plurality of peaks with an expected mass of the AFP glycoform to obtain an AFP glycoform peak and wherein determining the abundance of the AFP glycoform further comprises measuring an intensity of the AFP glycoform peak.

A method of identifying and determining an abundance of an AFP glycoform, wherein identifying the at least one AFP glycoform further comprises identifying a plurality of AFP core fucosylation (AFP-L3) glycoform peaks from the mass spectrum to obtain a plurality of AFP-L3 glycoform peaks and determining an abundance of the AFP glycoform further comprises measuring the intensity of the plurality of AFP-L3 glycoform peaks to obtain a total abundance of AFP-L3 glycoform in the biological sample.

A method of identifying and determining an abundance of an AFP glycoform, further including identifying a plurality of AFP core nonfucosylated (AFP-L1) glycoform peaks from the plurality of peaks of the mass spectrum to obtain a plurality of AFP-L1 glycoform peaks and determining an abundance of AFP-L1 glycoform by measuring an intensity of the plurality of AFP-L1 glycoform peaks to obtain a total abundance of AFP- L1 glycoform in the biological sample.

A method of identifying and determining an abundance of an AFP glycoform, further comprising determining a relative abundance of AFP-L3 glycoform in the biological sample from the total abundance of the AFP-L3 glycoform and the total abundance of the AFP-L1 glycoform according to the following formula

$$\frac{total abundance of AFP - L 3 glycoform}{\begin{matrix}total abundance of AFP - L 3 glycoform + \\ total abundance of AFP - L 1 glycoform\end{matrix}} .$$

A method of identifying and determining an abundance of an AFP glycoform, wherein separating the AFP from the biological sample further includes adding anti-AFP antibodies to the biological sample to form antibody-bound AFP, separating the antibody-bound AFP from the biological sample, and eluting the AFP from the anti-AFP antibodies as the enriched AFP sample.

A method of identifying and determining an abundance of an AFP glycoform, wherein separating the AFP from the biological sample further includes adding anti-AFP aptamers to the biological sample to form aptamer-bound AFP, separating the aptamer-bound AFP from the biological sample, and eluting the AFP from the anti-AFP aptamers as the enriched AFP sample.

A method of identifying and determining an abundance of an AFP glycoform, wherein the intact AFP is separated from the enriched AFP sample using high performance liquid chromatography.

A method of identifying and determining an abundance of an AFP glycoform, wherein the intact AFP is separated from the enriched AFP sample using capillary electrophoresis.

A method of identifying and determining an abundance of an AFP glycoform, wherein the high-resolution mass spectrometry is performed on the intact AFP protein with unmodified glycans.

What is claimed is:

1. A method of identifying an alpha fetoprotein (AFP) glycoform in a biological sample, comprising:
obtaining a biological sample;
separating AFP from the biological sample as an enriched AFP sample;
separating intact AFP from the enriched AFP sample;
subjecting the intact AFP to high resolution mass spectrometry to generate a mass spectrum; and
identifying an AFP glycoform from the mass spectrum.

2. The method of claim 1, wherein identifying the AFP glycoform further comprises deconvoluting the mass spectrum into a plurality of peaks and identifying the AFP glycoform by comparing a mass of at least one of the plurality of peaks with an expected mass of the AFP glycoform.

3. The method of claim 1, wherein separating the AFP from the biological sample further includes:
adding anti-AFP antibodies to the biological sample to form antibody-bound AFP;
separating the antibody-bound AFP from the biological sample; and
eluting the AFP from the anti-AFP antibodies as the enriched AFP sample.

4. The method of claim 3, wherein the anti-AFP antibodies are monoclonal antibodies.

5. The method of claim 1, wherein separating the AFP from the biological sample further includes:
adding anti-AFP aptamers to the biological sample to form aptamer-bound AFP;
separating the aptamer-bound AFP from the biological sample; and
eluting the AFP from the anti-AFP aptamers as the enriched AFP sample.

6. The method of claim 1, wherein the intact AFP is separated from the enriched AFP sample using high performance liquid chromatography.

7. The method of claim 1, wherein the intact AFP is separated from the enriched AFP sample using capillary electrophoresis.

8. The method of claim 1, wherein the high-resolution mass spectrometry is performed on the intact AFP with unmodified glycans.

9. The method of claim 1, wherein the AFP glycoform is a glycoform variant of core fucosylated (AFP-L3).

10. The method of claim 9, wherein the glycoform variant is selected from G1F, G1FS1, G2F, G2FS1, G2FS2, or a combination thereof.

11. The method of claim 1, wherein the AFP glycoform is a glycoform variant of core nonfucosylated (AFP-L1).

12. The method of claim 11, wherein the glycoform variant is selected from G1, G1S1, G2, G2S1, G2S2, or a combination thereof.

13. A method of identifying and determining an abundance of an alpha fetoprotein (AFP) glycoform in a biological sample, comprising:
obtaining a biological sample;
separating AFP from the biological sample as an enriched AFP sample;
separating intact AFP from the enriched AFP sample;
subjecting the intact AFP to high resolution mass spectrometry to generate a mass spectrum;
identifying at least one AFP glycoform from the mass spectrum; and
determining an abundance of the at least one AFP glycoform in the biological from the mass spectrum.

14. The method of claim 13, wherein identifying the at least one AFP glycoform from the mass spectrum further comprises deconvoluting the mass spectrum into a plurality of peaks and identifying the AFP glycoform by matching a mass of at least one of the plurality of peaks with an expected mass of the AFP glycoform to obtain an AFP glycoform peak and wherein determining the abundance of the AFP glycoform further comprises measuring an intensity of the AFP glycoform peak.

15. The method of claim 13, wherein identifying the at least one AFP glycoform further comprises identifying a plurality of AFP core fucosylation (AFP-L3) glycoform peaks from the mass spectrum to obtain a plurality of AFP-L3 glycoform peaks and determining an abundance of the AFP glycoform further comprises measuring the intensity of the plurality of AFP-L3 glycoform peaks to obtain a total abundance of AFP-L3 glycoform in the biological sample.

16. The method of claim 15, further comprising:
identifying a plurality of AFP core nonfucosylated (AFP-L1) glycoform peaks from the plurality of peaks of the mass spectrum to obtain a plurality of AFP-L1 glycoform peaks; and
determining an abundance of AFP-L1 glycoform by measuring an intensity of the plurality of AFP-L1 glycoform peaks to obtain a total abundance of AFP-L1 glycoform in the biological sample.

17. The method of claim 16, further comprising determining a relative abundance of AFP-L3 glycoform in the biological sample from the total abundance of the AFP-L3 glycoform and the total abundance of the AFP-L1 glycoform according to the following formula
$$\frac{total abundance of AFP - L 3 glycoform}{\begin{matrix}total abundance of AFP - L 3 glycoform + \\ total abundance of AFP - L 1 glycoform\end{matrix}} .$$

18. The method of claim 13, wherein separating the AFP from the biological sample further includes:
adding anti-AFP antibodies to the biological sample to form antibody-bound AFP;
separating the antibody-bound AFP from the biological sample; and
eluting the AFP from the anti-AFP antibodies as the enriched AFP sample.

19. The method of claim 13, wherein separating the AFP from the biological sample further includes:
adding anti-AFP aptamers to the biological sample to form aptamer-bound AFP;
separating the aptamer-bound AFP from the biological sample; and
eluting the AFP from the anti-AFP aptamers as the enriched AFP sample.

20. The method of claim 13, wherein the intact AFP is separated from the enriched AFP sample using high performance liquid chromatography.

21. The method of claim 13, wherein the intact AFP is separated from the enriched AFP sample using capillary electrophoresis.

22. The method of claim 13, wherein the high-resolution mass spectrometry is performed on the intact AFP protein with unmodified glycans.
